(19) United States
(12) Patent Application Publication
(10) Pub. No.: US 2025/0144377 A1
(43) Pub. Date: May 8, 2025

(54) MEDICAL INFUSION METHODS AND SYSTEMS

(71) Applicant: EDWARDS LIFESCIENCES CORPORATION, Irvine, CA (US)

(72) Inventors: Zachary Christopher Berwick, Tustin, CA (US); Yaeer E. Lev, Torrance, CA (US); Allen Jeong Keel, Irvine, CA (US)

(21) Appl. No.: 19/018,409

(22) Filed: Jan. 13, 2025

Related U.S. Application Data
(63) Continuation of application No. PCT/US2023/071028, filed on Jul. 26, 2023.
(60) Provisional application No. 63/370,432, filed on Aug. 4, 2022.

(51) Int. Cl. A61M 25/10; A61M 25/00

(52) U.S. Cl. CPC A61M 25/10; A61M 25/0097; A61M 2025/0004; A61M 2202/0007; A61M 2202/0468; A61M 2210/1082

(57) ABSTRACT

A method of infusing a solution to a target site in a patient is described. In one application, the method may be used for infusing a solution into a kidney. A cannula catheter includes a catheter shaft, the catheter shaft having a first lumen extending from a proximal end of the catheter shaft to a side opening along a distal portion. A cannula shaft of an infusion cannula can be pre-loaded within the first lumen. The method may include advancing the cannula shaft and the catheter shaft along a guidewire. The method can further include withdrawing the guidewire and advancing the cannula shaft relative to the catheter shaft for advancing a distal end of the cannula shaft through the side opening, thereby facilitating infusion of a solution from the cannula shaft to the target site.

### CROSS REFERENCE TO RELATED APPLICATION(S)

This application is a continuation of International Application No. PCT/US2023/071028, filed Jul. 26, 2023, which claims the benefit of U.S. Provisional Patent Application No. 63/370,432, filed Aug. 4, 2022, the disclosures of which are hereby incorporated by reference.

### BACKGROUND

The present disclosure generally relates to the field of minimally invasive percutaneous transcatheter delivery of medical devices and/or therapies. Minimally invasive percutaneous transcatheter medical procedures can comprise providing a medical infusion fluid to a target location, including a target location in a kidney. Delivery of the medical infusion fluid to the target location can be used to treat a variety of conditions, including conditions of the kidney and/or heart.

### SUMMARY

Described herein are methods, devices and/or systems relating to transvascularly providing a medical infusion fluid to a target site within an inner medulla of a kidney. The medical infusion fluid can comprise a solution having one or more oncotically active agents. The solution comprising the one or more oncotically active agents can be provided into interstitium tissue within the inner medulla at the target site to modify an osmotic pressure within the interstitium tissue at the target site. A vascular wall portion at a target site can be punctured such that the infusion fluid can be delivered therethrough into the interstitium tissue surrounding, and/or adjacent and/or proximate to the target site. The inner medulla can be transvascularly accessed through the renal vein. The target site can be in a blood vessel that is a direct or indirect branch of the renal vein, including a venule vessel, in the inner medulla. The target site can be selected so as to provide the infusion fluid to interstitium tissue surrounding, adjacent to and/or proximate to a loop of Henle in the inner medulla.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1A shows an example of a medical infusion system disposed through a femoral vein and along a transvascular delivery path for minimally invasive percutaneous transcatheter delivery of an infusion fluid to a target inner renal medulla location, in accordance with one or more examples.

FIG. 1B provides a detailed view of the target inner renal medulla location described with reference to FIG. 1A in accordance with one or more examples.

FIGS. 2A and 2B provide perspective and longitudinal cross-sectional views, respectively, of a distal portion of the medical infusion system described with reference to FIGS. 1A and 1B, in accordance with one or more examples.

FIG. 3A provides a longitudinal cross-sectional view of the distal portion of the medical infusion system described with reference to FIGS. 1A, 1B, 2A and 2B, where the cannula shaft is preloaded within the catheter shaft and a guidewire disposed therethrough, in accordance with one or more examples.

FIGS. 3B and 3C provide a longitudinal cross-sectional view and a perspective view, respectively, of the distal portion of the medical infusion system described with reference to FIG. 3A, where a portion of the cannula shaft has been advanced through the side opening of the catheter shaft, in accordance with one or more examples.

FIG. 4 provides a side view of a distal portion of a cannula shaft in accordance with one or more examples.

FIGS. 5A, 5B, 5C and 5D show various steps of an example of a process for providing an infusion to a target site within an inner renal medulla using the medical infusion system described with reference to FIGS. 1A, 1B, 2A and 2B, where a delivery catheter is used to facilitate positioning of the medical infusion system, in accordance with one or more examples.

FIG. 6 is a process flow diagram of an example of a process for transvascularly providing an infusion to a target inner renal medulla location using a medical infusion system as described herein, in accordance with one or more examples.

FIG. 7 is a process flow diagram of an example of a process for transvascularly providing an infusion to a target location within an inner renal medulla, in accordance with one or more examples.

### DETAILED DESCRIPTION

The headings provided herein are for convenience only and do not necessarily affect the scope or meaning of the claimed invention.

Although certain preferred examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

In some cases, increases in central venous pressure during heart failure can lead to renal venous congestion. The presence of chronic kidney disease (CKD) can contribute to this effect. This effect can further contribute to renal dysfunction and create a positive feedback loop. For example, impaired renal diuretic efficacy can promote fluid retention, which can exacerbate the renal venous congestion and lead to more fluid retention. This cycle of fluid retention can continue until acute cardiac decompensation occurs. Driving forces for filtration into the kidney can comprise a difference between arterial and venous pressures. A decrease in renal filtration gradient can lead to increased fluid retention and thereby contribute to renal venous congestion. Reduced renal functional reserve can hinder the ability of the kidney from adequately responding to changes in venous pressure. Patients who have chronic kidney disease can be more sensitive to changes in blood volume and/or be more prone to blood volume overload, thereby leading to repeat hospitalizations for acute decompensated heart failure.

Described herein are methods, devices and/or systems relating to transvascularly accessing a kidney to provide a transvascular access site in an inner medulla of a kidney so as to provide a medical infusion fluid to interstitium tissue in the inner medulla. The infusion fluid can comprise a solution comprising an oncotically active agent. A vascular wall portion at a target site, such as the transvascular access site, can be punctured such that the infusion fluid can be delivered therethrough into interstitium tissue surrounding, and/or adjacent and/or proximate to the target site. In some instances, the inner medulla can be accessed through the renal vein. In some instances, the target site can be in a blood vessel that is direct or indirect branch of the renal vein. In some instances, the target site can be in a venule vessel in the inner medulla. In some instances, the target site can be selected so as to provide the infusion fluid to interstitium tissue surrounding and/or adjacent and/or proximate to a loop of Henle in the inner medulla.

Infusing into tissue of the inner renal medulla can be configured to modify the osmotic pressure within the inner renal medulla, including osmotic pressure within the inner medulla surrounding and/or adjacent and/or proximate to the loop of Henle. Modifying the osmotic pressure within the inner medulla of the kidney can advantageously decrease water reabsorption in the kidney. A decrease in reabsorption of water can lead to an increase in urine output and a decrease in blood volume. Decreased blood volume can lead to reduced renal venous pressure to facilitate an increase in the renal pressure gradient. Increasing the renal pressure gradient can improve renal filtration function. Improved renal filtration function can facilitate improvement in acute decompensated heart failure (ADHF), such as in cardiorenal syndrome patients. The infusion therapy described herein can advantageously reduce renal venous pressure to facilitate increased renal pressure gradient, thereby improving filtration function to reduce blood volume and fluid retention.

A medical infusion system as described herein can be used to provide the desired infusion to the inner medulla. In some instances, the medical infusion system can comprise a cannula catheter comprising a catheter shaft extending distally from a catheter hub. The medical infusion system can comprise an infusion cannula that includes a cannula shaft extending distally from a cannula hub. The cannula shaft can comprise an infusion lumen extending therethrough. At least a portion of the cannula shaft can be configured to be preloaded within a first lumen portion of a catheter lumen of the catheter shaft. The first lumen portion can extend from a proximal end to a side opening on a distal portion of the catheter shaft. The catheter lumen can comprise a second lumen portion disposed distally of and that is in fluid communication with the first lumen portion, for example extending from the first lumen portion to a distal end of the catheter shaft. A distal end of the cannula shaft can be adjacent to the side opening of the catheter shaft, for example disposed within the first lumen portion and adjacent to the side opening, while the cannula shaft is preloaded therein. The catheter shaft carrying the cannula shaft preloaded therein can be advanced along a guidewire to a target site in the inner medulla. For example, a portion of the guidewire can be slidably disposed within the infusion lumen of the cannula shaft and another portion of the guidewire can be slidably disposed within the second lumen portion of the catheter shaft. After a distal portion of the catheter shaft is desirably positioned in the inner medulla, the guidewire can be withdrawn such that a portion of the cannula shaft can be advanced through the side opening of the catheter shaft. The distal end of the cannula shaft can be positioned through a wall portion of the blood vessel and into interstitium tissue adjacent to the blood vessel to allow delivery of the infusion fluid into the interstitium tissue.

Although the medical infusion systems described herein are described as being inserted into the femoral vein for delivery of an infusion fluid to the kidney, it will be understood that the medical infusion systems can be inserted into any number of other vessels and/or lumens. In some alternative instances, the medical infusion systems can be inserted into any number of other vessels and/or lumens for minimally invasive transcatheter delivery of an infusion fluid to another target location and/or via other delivery pathways. It will be understood that one or more components of the medical infusion systems can undergo various processes in preparation for their use in the procedures, including for example sterilization processes. The medical infusion systems can be sterilized medical infusion systems. For example, cannula catheters and/or infusion cannulae as described herein can be sterilized cannula catheters and/or sterilized infusion cannulae.

As used herein, a “delivery lumen” can refer to any number of lumens, channels, passages, and/or conduits through which a medical instrument and/or medical device can be advanced, including for example a working channel. For example, a “delivery lumen” as used herein can refer to a working channel through which a medical instrument and/or medical device can be advanced for positioning the medical instrument and/or medical device at a target site within a patient.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

It will be understood that one or more components of the medical access sheaths, catheters, cannulae and/or systems can undergo various processes in preparation for their use in the procedures, including for example sterilization processes. The medical access sheaths, catheters, cannulae and/or systems can be sterilized medical access sheaths, catheters, cannulae and/or systems. Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

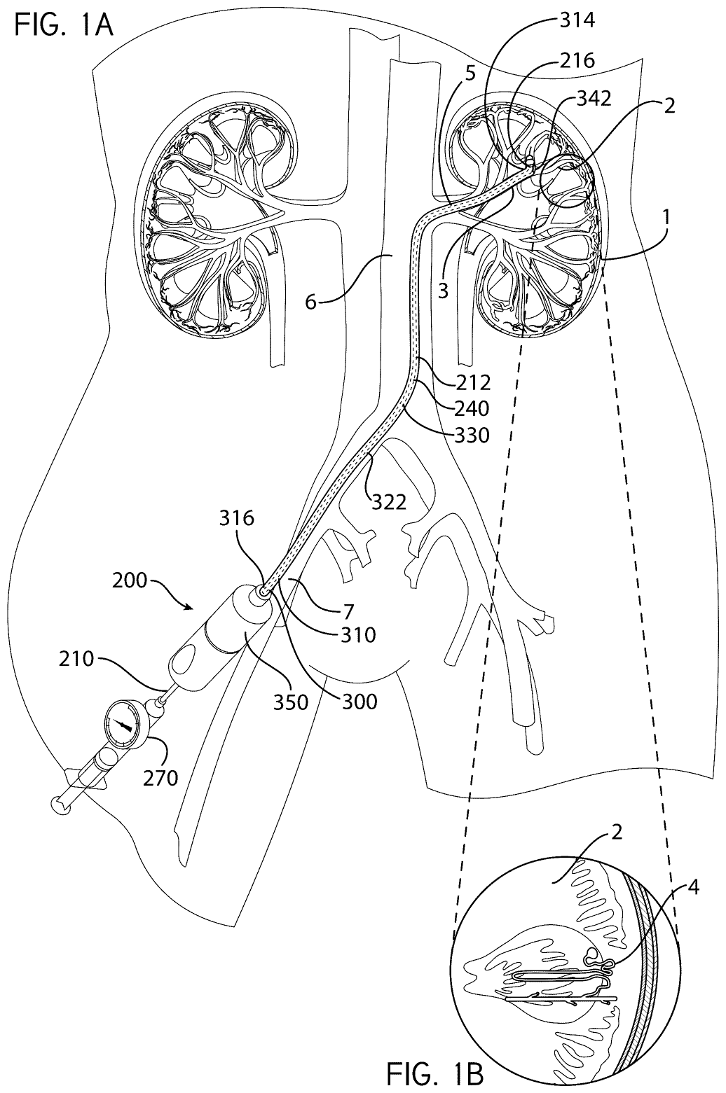

FIG. 1A shows an example of a medical infusion system 200 comprising a portion disposed through a femoral vein 7 for minimally invasive percutaneous transcatheter delivery of an infusion fluid to a target location in an inner medulla 2 of a kidney 1. The medical infusion system 200 can comprise a cannula catheter 300 configured to slidably receive a portion of an infusion cannula 210. For example, the cannula catheter 300 can comprise a catheter hub 350 and a catheter shaft 310 extending distally from the catheter hub 350. The catheter hub 350 can comprise a catheter hub lumen 360 (not shown) extending therethrough. The catheter shaft 310 can have a catheter lumen 322 extending therethrough. The catheter lumen 322 can comprise a first lumen portion 330 extending from a proximal end 316 of the catheter shaft 310 to a side opening 342 on a distal portion 314 of the catheter shaft 310. The infusion cannula 210 can comprise a cannula shaft 212 having an infusion lumen 240 extending therethrough. An infusion fluid can be delivered to a target site through the infusion lumen 240. At least a portion of the cannula shaft 212 can be configured to be slidably disposed in the first lumen portion 330.

In some instances, the infusion cannula 210 can be preloaded within the cannula catheter 300 while the cannula catheter 300 is advanced to a desired location in the kidney 1. For example, at least a portion of the catheter shaft 310 carrying the cannula shaft 212 can be inserted through an access opening formed on the femoral vein 7. The catheter shaft 310 can be advanced through the femoral vein 7 and the inferior vena cava 6, and into the renal vein 5. A portion of the catheter shaft 310 can be positioned into the renal vein 5. For example, the distal portion 314 of the catheter shaft 310 can be positioned into the renal vein 5, and from the renal vein 5 into a blood vessel that is a direct or indirect branch of the renal vein 5. In some instances, a portion, such as the distal portion 314, of the catheter shaft 310 can be positioned at a target location in a blood vessel that is a direct or indirect branch of the renal vein 5. The target location can be in a blood vessel within the inner medulla 2. In some instances, a portion, such as the distal portion 314, of the catheter shaft 310 can be positioned at a target location in a venule vessel 3. After the distal portion 314 of the catheter shaft 310 is positioned at a desired location, such as a location within the venule vessel 3, a portion of the cannula shaft 212 can be advanced out of the first lumen portion 330 through the side opening 342. As described in further detail herein, a distal end 220 (not shown) of the cannula shaft 212 can puncture a wall portion of the venule vessel 3 as the portion of the cannula shaft 212 is advanced out of the side opening 342 and positioned into the interstitium tissue of the kidney 1, such as interstitium tissue of the inner medulla 2. For example, a distal portion 216 of the cannula shaft 212 can be advanced through the side opening 342. An infusion fluid can be provided through the cannula shaft 212 after the cannula shaft 212 is deployed.

Referring to FIG. 1B, a target area to which the infusion fluid is provided can be in the inner medulla 2. In some instances, the infusion fluid can be delivered to interstitium tissue in the inner medulla 2. In some instances, the infusion fluid can be provided to interstitium tissue in the inner medulla 2 that surround, and/or are proximate and/or adjacent to, the loop of Henle 4. For example, target tissue into which the infusion fluid is provided can comprise interstitium tissue in the inner medulla 2 that surround, and/or are proximate and/or adjacent to, the loop of Henle 4. The infusion fluid can comprise a solution having one or more oncotically active agents. The solution can be delivered to the target inner renal medulla 2 location to alter the osmotic gradient in the inner medulla 2, so as to reduce water reabsorption within the kidney 1. A solution comprising one or more oncotically modifying agents can be delivered to the inner renal medulla 2 to decrease reabsorption of water, thereby increasing urine output and decreasing blood volume. Decreased blood volume can lead to reduced renal venous pressure. Reducing renal venous pressure can facilitate improved renal pressure gradient, which can thereby facilitate improved filtration function. Patients who demonstrate resistance to diuretic therapy can benefit from the infusion therapy.

Typically, blood vessels adjacent to the loop of Henle 4 can have oncotically concentrated blood to serve as a driving force for water reabsorption from the loop of Henle 4. For example, the vasa recta and peritubular capillaries can typically contain oncotically concentrated blood, which can serve as a driving force for water reabsorption from the loop of Henle 4. If the osmotic pressure in the interstitium surrounding, and/or adjacent and/or proximate to, the loop of Henle 4 is increased, for example raised to above that of the blood in this region, then reabsorption of water from the loop of Henle 4 would be prevented or decreased. If osmotic pressure in that interstitium tissue is increased, including being raised to above that of the loop of Henle 4, then transport into the interstitial space for subsequent reabsorption would be prevented or decreased.

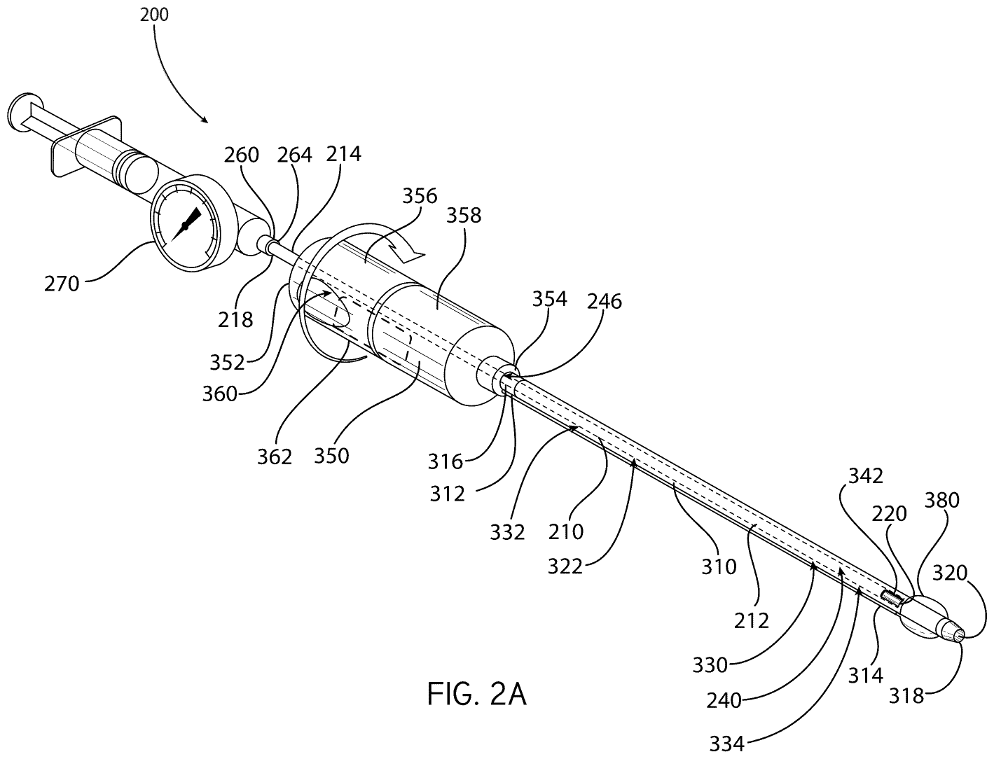

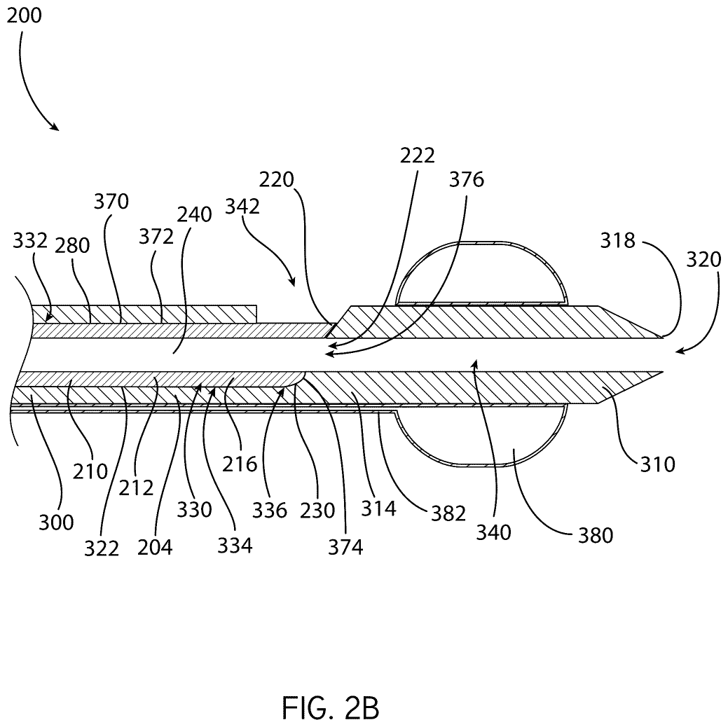

FIG. 2A is a perspective view of the medical infusion system 200 and FIG. 2B is a longitudinal cross-sectional view of a distal portion 204 of the medical infusion system 200. As described in further detail herein, the medical infusion system 200 can be configured to be transvascularly advanced into an inner renal medulla to deliver a solution comprising one or more oncotically active agents. FIGS. 2A and 2B show the infusion cannula 210 preloaded within the cannula catheter 300. A portion of the cannula shaft 212 is shown as being preloaded within the cannula catheter 300. The cannula catheter 300 can comprise the catheter hub 350 coupled to a proximal portion 312 of the catheter shaft 310. For example, the catheter shaft 310 can extend from a distal end 354 of the catheter hub 350. The catheter hub 350 can comprise a catheter hub lumen 360 extending therethrough, for example from a proximal end 352 to the distal end 354 of the catheter hub 350. The catheter shaft 310 can have a catheter lumen 322 extending therethrough. An inflatable balloon 380 can be associated with, such as coupled to, a distal portion 314 of the catheter shaft 310. Respective portions of the cannula shaft 212 can be disposed through the catheter hub lumen 360 and at least a portion of the first lumen portion 330 of the catheter lumen 322. The first lumen portion 330 can extend from the side opening 342 and through portions of the catheter shaft 310 proximal of the side opening 342, for example extending from the proximal end 316 of the catheter shaft 310 to the side opening 342 on the distal portion 314 of the catheter shaft 310. In some instances, the proximal end 314 of the catheter shaft 310 can be adjacent and/or coupled to the distal end 354 of the catheter hub 350. A proximal portion 214 of the cannula shaft 212 can extend proximally of the catheter hub 350, for example extending proximally of the proximal end 352 of the catheter hub 350.

The catheter hub 350 can comprise a cannula advancement assembly 362 configured to control translational movement of the infusion cannula 210. For example, the cannula advancement assembly 362 can be configured to engage with the infusion cannula 210 to control translational movement of the infusion cannula 210 relative to the cannula catheter 300. The cannula advancement assembly 362 can be configured to engage with a corresponding portion of the cannula shaft 212 extending through the catheter hub lumen 360 so as to control translational movement of the infusion cannula shaft 212 relative to the cannula catheter 300. The catheter hub 350 can comprise a first catheter hub portion 356 and a second catheter hub portion 358. The first catheter hub portion 356 can be rotatable around a longitudinal axis of the catheter hub 350 relative to the second catheter hub portion 358. The first catheter hub portion 356 is shown as being disposed proximally relative to the second catheter hub portion 358. In some instances, rotation of the first catheter hub portion 356 relative to the second catheter hub portion 358 in a first direction can be configured to cause the cannula advancement assembly 362 to translate the cannula shaft 212 distally relative to the cannula catheter 300, including the catheter shaft 310. Rotation of the first catheter hub portion 356 relative to the second catheter hub portion 358 in a second opposing direction can be configured to cause the cannula advancement assembly 362 to translate the cannula shaft 212 proximally relative to the cannula catheter 300, including the catheter shaft 310.

The infusion cannula 210 can comprise a cannula hub 260 having a hub lumen extending therethrough. The infusion cannula 210 can comprise the cannula shaft 212 extending distally from the cannula hub 260, such as from a distal end 264 of the cannula hub 260. The hub lumen can be in fluid communication with the infusion lumen 240 extending through the cannula shaft 212. The infusion lumen 240 can extend from a proximal end 218 of the cannula shaft 212 to the distal end 220 of the cannula shaft 212. For example, an infusion fluid can be delivered through the hub lumen and the infusion lumen 240 to a target site. The cannula hub 260 can be disposed proximally of the catheter hub 350, such as while the infusion cannula 210 is preloaded within the cannula catheter 300.

In some instances, a syringe 270 can be configured to be coupled to the infusion cannula 210 to deliver the infusion fluid into and/or control fluid flow into the infusion cannula 210. The syringe 270 can be coupled to the cannula hub 260. For example, the infusion fluid can be delivered through the hub lumen and the infusion lumen 240. In some instances, the syringe 270 can comprise a dial syringe. The dial syringe can be configured to provide desired control of the fluid flow through the infusion cannula 210. For example, the dial syringe can comprise a dial valve configured to control fluid flow therethrough, thereby controlling the fluid flow into the infusion cannula 210.

Referring to FIG. 2B, the catheter lumen 322 can comprise the first lumen portion 330. As described herein, the first lumen portion 330 can extend from a proximal end 316 to the side opening 342 on the distal portion 314 of the catheter shaft 310. The second lumen portion 340 can be disposed distally of and be in fluid communication with the first lumen portion 330. A proximal portion 332 of the first lumen portion 330 can extend along a dimension parallel or substantially parallel to a longitudinal axis of the catheter shaft 310. In some instances, the proximal portion 332 can be coaxial with the longitudinal axis of the catheter shaft 310. A portion of the first lumen portion 330 can comprise a curved portion 336 between the portion of the first lumen portion 330 extending along the dimension parallel to and/or coaxial with the longitudinal axis of the catheter shaft 310 and the side opening 342. The curved portion 336 can comprise at least a portion that follows a curved and/or arcuate path. A distal portion 334 of the first lumen portion 330 can comprise the curved portion 336. In some instances, the curved portion 336 can extend from the portion of the first lumen portion 330 extending along the dimension parallel to and/or coaxial with the longitudinal axis of the catheter shaft 310 to the side opening 342. For example, an inner lumen wall 370 defining the first lumen portion 330 can comprise a coaxial inner lumen wall portion 372 defining the dimension parallel to and/or coaxial with the longitudinal axis of the catheter shaft 310. The inner lumen wall 370 can comprise a curved portion 374 between the coaxial inner lumen wall portion 372 and the side opening 342 on the catheter shaft 310 to define the curved portion 336 of the first lumen portion 330. In some instances, the curved portion 374 can extend from the coaxial inner lumen wall portion 372 to the side opening 342 on the catheter shaft 310 to define the curved portion 336 of the first lumen portion 330. For example, the curved portion 374 can be adjacent to the side opening 342. The curved portion 374 can comprise at least a portion that defines a curved and/or arcuate path. The curved portion 374 can provide a ramp and/or slope configured to guide an exit trajectory of the cannula shaft 212 as the cannula shaft 212 is deployed out of the first lumen portion 330 through the side opening 342. The curved portion 374 of the inner lumen wall 370 can comprise an opening 376 extending therethrough such that the first lumen portion 330 is in fluid communication with the second lumen portion 340. For example, the second lumen portion 340 can extend from the opening 376 to the distal end 318 of the catheter shaft 310. The catheter shaft 310 can comprise a distal opening 320 at the distal end 318 of the catheter shaft 310 that is in fluid communication with the second lumen portion 340. As described in further detail herein, a guidewire configured to be slidably disposed through the medical infusion system 200 can be configured to extend through the opening 376 on the curved portion 374 and the distal opening 320 such that the medical infusion system 200 can be advanced along the guidewire to the target location.

While the cannula shaft 212 is preloaded within the cannula catheter 300, at least a portion of the cannula shaft 212 can be configured to be slidably disposed in the first lumen portion 330. To deploy the cannula shaft 212 from the cannula catheter 300, a portion of the cannula shaft 212 can be configured to be advanced out of the first lumen portion 330 through the side opening 342. In some instances, a distal portion 216 of the cannula shaft 212 can be configured to be advanced out of the first lumen portion 330 through the side opening 342.

While at least a portion of the cannula shaft 212 is preloaded within the first lumen portion 330, portions of the cannula shaft 212 within the first lumen portion 330 can extend along a dimension parallel or substantially parallel to a longitudinal axis of the catheter shaft 310. In some instances, the portions of the cannula shaft 212 can extend along a dimension coaxial with the longitudinal axis of the catheter shaft 310. In some instances, while at least a portion of the cannula shaft 212 is preloaded within the first lumen portion 330, the portion of the infusion lumen 240 within the catheter shaft 310 can be configured to be parallel or substantially parallel to and/or coaxial with the portion of the first lumen portion 330 extending along the dimension parallel to and/or coaxial with the longitudinal axis of the catheter shaft 310. For example, the portion of the infusion lumen 240 can be parallel or substantially parallel to and/or coaxial with the proximal portion 332 of the first lumen portion 330. In some instances, the longitudinal axes of the infusion lumen 240 and the proximal portion 332 of the first lumen portion 330 can be coaxial with the longitudinal axis of the cannula catheter 300.

In some instances, at least a portion of an externally oriented surface 280 of the cannula shaft 212 can be configured to be in contact with a corresponding portion of the inner lumen wall 370 defining the first lumen portion 330. In some instances, externally oriented surface portions 280 of the cannula shaft 212 received by the first lumen portion 330 can be configured to be in contact with corresponding portions of the inner lumen wall 370 defining the first lumen portion 330. For example, an outer width, such as an outer diameter, of the cannula shaft 212 and an inner width, such as an inner diameter, of the catheter shaft 310 (e.g., a width, such as a diameter, of the first lumen portion 330) can be selected to provide desired contact between the externally oriented surface portions 280 of the cannula shaft 212 and the inner lumen wall 370. In some instances, an outer diameter of the cannula shaft 212 can be the same as and/or similar to a diameter of the first lumen portion 330. In some instances, the diameter of the first lumen portion 330 can be uniform or substantially uniform along an entire length thereof. In some instances, an outer diameter of the cannula shaft 212 can be uniform or substantially uniform along an entire length thereof, such as an entire length of the cannula shaft 212 configured to be slidably disposed within the first lumen portion 330. The diameter of the first lumen portion 330 and the outer diameter of the cannula shaft 212 can be the same such that the externally oriented surface portions 280 of the cannula shaft 212 can maintain contact with the inner lumen wall 370 while the cannula shaft 212 is slidably disposed therethrough. Contact between the cannula shaft 212 and the inner lumen wall 370 can facilitate desired control in the orientation of the cannula shaft 212 and/or rate at which the cannula shaft 212 is advanced when advancing the cannula shaft 212 to deploy the distal end 220 of the cannula shaft 212 through the side opening 342 of the catheter shaft 310.

In some instances, the distal portion 216 of the cannula shaft 212 can comprise a curved and/or rounded portion 230. As described herein, the cannula shaft 212 can comprise the distal opening 222 at the distal end 220 thereof and in fluid communication with the infusion lumen 240. The curved and/or rounded portion 230 can be adjacent to the distal opening 222. In some instances, while the cannula shaft 212 is preloaded within the first lumen portion 330, the curved and/or rounded portion 230 can be configured to be positioned against the curved portion 374 of the inner lumen wall 370. The curvature and/or rounded shape of the curved and/or rounded portion 230 can be configured to facilitate deployment of the cannula shaft 212 through the side opening 342 on the catheter shaft 310. In some instances, the curved and/or rounded portion 230 can facilitate its advancement along the curved portion 374 of the inner lumen wall 370, thereby facilitating advancement of a portion of the cannula shaft 212 through the side opening 342.

The opening 376 on the curved portion 374 of the inner lumen wall 370 can be configured to be aligned with the distal opening 222 of the cannula shaft 212 while the cannula shaft 212 is preloaded within the first lumen portion 330. Alignment of the openings 376, 222 can facilitate extension therethrough of the guidewire such that the guidewire can pass from the infusion lumen 240 of the cannula shaft 212 into the second lumen portion 340 of the catheter shaft 310. In some instances, the opening 376 on the curved portion 374 of the inner lumen wall 370 can be on the longitudinal axis of the cannula catheter 300. While the cannula shaft 212 is preloaded within the first lumen portion 330, the distal opening 222 can be on a longitudinal axis of the cannula catheter 300.

In some instances, while the cannula shaft 212 is preloaded within the first lumen portion 330, the distal end 220 of the cannula shaft 212 can be adjacent or proximate to the side opening 342 on the catheter shaft 310. In some instances, the distal end 220 of the cannula shaft 212 can comprise at least a portion that is configured to pierce tissue. For example, the distal end 220 can be configured to pierce a wall portion of a blood vessel into which the cannula shaft 212 is advanced such that a desired portion of the cannula shaft 212 can be positioned through the blood vessel wall to allow infusion to tissue adjacent to the blood vessel. For example, the distal end 220 can be a sharp distal end configured to pierce tissue. The distal end 220, including the sharp distal end, can be configured to be disposed within the catheter lumen 322 while the cannula shaft 212 is preloaded within the catheter lumen 322. In some instances, the distal end 220 can be adjacent to the side opening 342 of the catheter shaft 310. The distal end 220 can be configured to remain within the catheter lumen 322, such as the first lumen portion 330, to prevent premature piercing of tissue by the distal end 220, such as during delivery of the infusion system 200 to a target location.

In some instances, the sharp distal end can be opposingly oriented around a circumference of the cannula shaft 212 relative to the curved and/or rounded portion 230. For example, the sharp distal end can be oriented such that as the cannula shaft 212 is advanced out of the side opening 342 of the catheter shaft 310 such that the sharp distal end can contact and pierce the target tissue and the curved and/or rounded portion 230 can glide along the curved portion 374 of the inner lumen wall 370.

Referring again to FIG. 2B, the cannula catheter 300 can comprise the catheter shaft 310 having the catheter lumen 322 extending therethrough. The catheter lumen 322 can comprise the second lumen portion 340 extending distally from the first lumen portion 330 to the distal end 318 of the catheter shaft 310. As described herein, the catheter shaft 310 can comprise the distal opening 320 at the distal end 318 of the catheter shaft 310 that is in fluid communication with the second lumen portion 340. While the cannula shaft 212 is preloaded within the first lumen portion 330, a first portion of a guidewire can be configured to be slidably disposed in the infusion lumen 240 of the cannula shaft 212. The second lumen portion 340 can be configured to slidably receive a corresponding portion of the guidewire, such as a second portion of the guidewire. A corresponding portion of the guidewire can be directly disposed in the second lumen portion 340. For example, the guidewire can comprise a portion configured to be slidably disposed within the infusion lumen 240, such as from a proximal end 246 of the infusion lumen 240, and extend through the distal opening 222 on the cannula shaft 212. The guidewire can pass through the opening 376 on the curved portion 374 of the inner lumen wall 370 defining the first lumen portion 330 and extend into the second lumen portion 340. The guidewire can extend out of the second lumen portion 340 through the distal opening 320.

In some instances, the second lumen portion 340 can comprise a diameter smaller than that of the first lumen portion 330. The first lumen portion 330 can be sized to receive the cannula shaft 212. The second lumen portion 340 can be sized to receive the guidewire. In some instances, a portion of a wall of the catheter shaft 310 defining the second lumen portion 340 can be thicker than that of the wall of the catheter shaft 310 defining the first lumen portion 330. For example, the outer diameter of the catheter shaft 310 can be the same along an entire or substantially entire length of the catheter shaft 310. A thickness of the wall of the catheter shaft 310 defining the second lumen portion 340 can be thicker than that of the wall of the catheter shaft 310 defining the first lumen portion 330 such that the second lumen portion 340 can comprise a diameter smaller than that of the first lumen portion 330.

In some instances, an outer diameter of the catheter shaft 310 can be selected to facilitate its positioning into target blood vessels. As described herein, in some instances, target blood vessels can be a blood vessel in the kidney, including in the inner medulla. The outer diameter of the catheter shaft 310 can be dimensioned for positioning into a blood vessel of the kidney, including the inner medulla. In some instances, the outer diameter of the catheter shaft 310 can be less than about 2 French (Fr).

As described herein, an inflatable balloon 380 can be associated with, such as coupled to, the distal portion 314 of the catheter shaft 310. The inflatable balloon 380 can be coupled to an externally oriented surface portion on the distal portion 314 of the catheter shaft 310. In some instances, the inflatable balloon 380 can be around a circumference of the distal portion 314 of the catheter shaft 310. For example, the inflatable balloon 380 can assume a ring shape around the circumference of the distal portion 314. FIGS. 2A and 2B show the inflatable balloon 380 disposed distally of the side opening 342 on the catheter shaft 310. Alternatively, the inflatable balloon 380 can be disposed proximally of the side opening 342 on the catheter shaft 310. An inflation lumen 382 can extend along a length of the catheter shaft 310 and in fluid communication with the inflatable balloon 380 to facilitate inflation and/or deflation of the inflatable balloon 380. In some instances, at least a portion of the inflation lumen 382 can extend along a wall portion of the catheter shaft 310, for example being defined at least partially by an inflation wall portion extending along the wall portion of the catheter shaft 310. In some instances, the inflation lumen 382 can extend within a wall portion of the catheter shaft 310. For example, the wall portion of the catheter shaft 310 can comprise a portion that defines the inflation lumen 382.

The cannula shaft 212 can be made of any number of different types of materials. The cannula shaft 212 be made of material configured to provide mechanical strength along the longitudinal axis of the cannula shaft 212 to facilitate advancement of the cannula shaft 212 through tortuous anatomical pathways, while comprising flexibility to allow desired deployment out of the catheter shaft 310. In some instances, the cannula shaft 212 can comprise stain-less steel. In some instances, the cannula shaft 212 can be a stainless-steel infusion cannula. In some instances, the catheter shaft 310 can comprise a polymeric material. For example, the catheter shaft 310 can be a polymeric catheter.

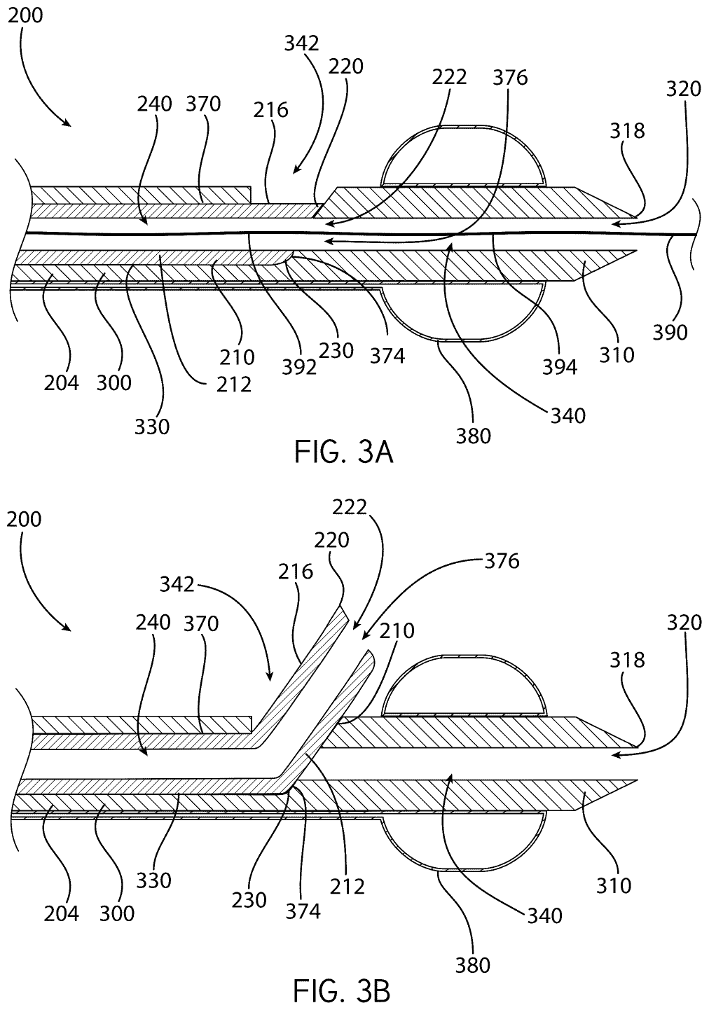

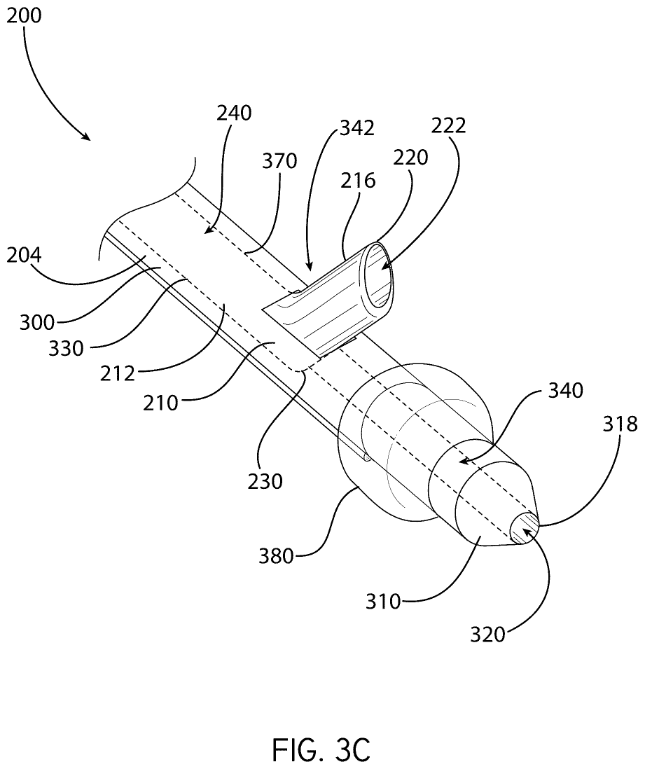

FIGS. 3A, 3B and 3C show various steps in a process to deploy a portion of the cannula shaft 212 through the side opening 342 of the catheter shaft 310. FIG. 3A is a longitudinal cross-sectional view of the distal portion 204 of the medical infusion system 200 comprising the cannula shaft 212 preloaded within the first lumen portion 330 of the catheter shaft 310 and a guidewire 390 disposed therethrough. FIG. 3B is a longitudinal cross-sectional view, and FIG. 3C is a perspective view, of the distal portion 204 of the medical infusion system 200 where the guidewire 390 has been withdrawn and the portion of the cannula shaft 212 has been advanced through the side opening 342 and out of the first lumen portion 330 of the catheter shaft 310. Referring to FIG. 3A, the medical infusion system 200 can be advanced along the guidewire 390 to a target location. The guidewire 390 can comprise a first portion 392 configured to be slidably disposed within the infusion lumen 240 of the cannula shaft 212. The first portion 392 of the guidewire 390 can extend from a proximal end 246 of the infusion lumen 240 to the distal opening 222 on the cannula shaft 212 and through the distal opening 222. The guidewire 390 can pass through the opening 376 on the curved portion 374 of the inner lumen wall 370 defining the first lumen portion 330 of the catheter shaft 310 and extend into the second lumen portion 340. The guidewire 390 can comprise a second portion 394 extending through the second lumen portion 340 of the catheter shaft 310. The guidewire 390 can extend out of the second lumen portion 340 through the distal opening 320. The catheter shaft 310 carrying the cannula shaft 212 can be advanced along the guidewire 390 to a target location.

After the medical infusion system 200 is advanced to the target location, the guidewire 390 can be withdrawn. Referring to FIGS. 3B and 3C, the guidewire 390 has been withdrawn from within the second lumen portion 340 and the infusion lumen 240. The infusion cannula 210 can then be deployed from the cannula catheter 300. For example, the cannula shaft 212 can be advanced relative to the catheter shaft 310 to position a distal end 220 of the cannula shaft 212 externally of the first lumen portion 330 through the side opening 342. A portion of the cannula shaft 212, including a distal portion 216, can be advanced out of the side opening 342.

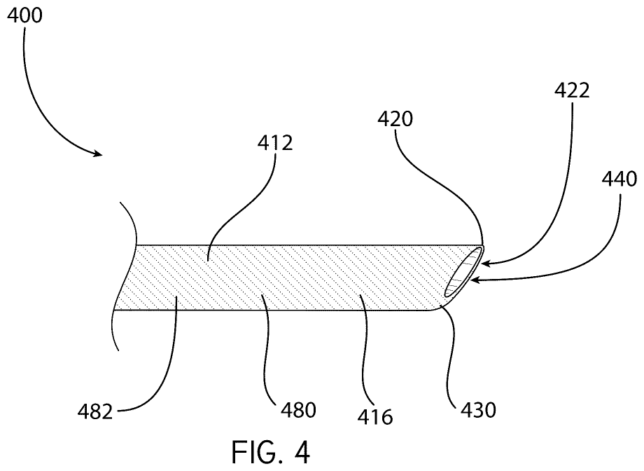

FIG. 4 is a side view of an example of a cannula shaft 412 of an infusion cannula 400. A distal portion 416 of the cannula shaft 412 is shown. The cannula shaft 412 can comprise an externally oriented surface 480 having a plurality of patterned indentations 482 on at least a portion thereof. In some instances, at least a portion of an externally oriented surface 480 of the cannula shaft 412 can be configured to be in contact with a corresponding portion of an inner lumen wall of a catheter shaft in which the cannula shaft 412 is disposed. For example, the externally oriented surface 480 of the cannula shaft 412 can maintain contact with the inner lumen wall defining a first lumen portion of the catheter shaft while the cannula shaft 412 is slidably disposed therethrough. For example, an outer diameter of the cannula shaft 412 can be the same as and/or similar to a diameter of the first lumen portion such that the externally oriented surface 480 of the portion of the cannula shaft 412 received by the first lumen portion can be configured to be in contact with corresponding portions of the inner lumen wall defining the first lumen portion. In some instances, the plurality of patterned indentations 482 can provide desired contact between the externally oriented surface 480 of the cannula shaft 412 and the inner lumen wall of the catheter shaft. In some instances, the plurality of patterned indentations 482 can be on an entirety or substantially entirety of the externally oriented surface 480 of the cannula shaft 412. In some instances, a pattern, directionality and/or shape of the plurality of patterned indentations 482 can facilitate desired contact between the externally oriented surface 480 of the cannula shaft 412 and the inner lumen wall of the catheter shaft to provide desired control in the orientation of the cannula shaft 412 and/or rate at which the cannula shaft 412 is advanced through the side opening of the catheter shaft. The plurality of patterned indentations 482 can comprise any number of one or more of different types of depressions, grooves, and/or recesses.

The infusion cannula 400 can comprise one or more other features of the infusion cannula 210 described with reference to FIGS. 1A, 1B, 2A, 2B, 3A, 3B and 3C. For example, the cannula shaft 412 can comprise an infusion lumen 440 extending therethrough. The cannula shaft 412 can comprise a distal opening 422 at a distal end 420. The distal opening 422 can be in fluid communication with the infusion lumen 440. The distal portion 416 of the cannula shaft 412 can comprise a curved and/or rounded portion 430. The curved and/or rounded portion 430 can be adjacent to the distal opening 422. In some instances, the distal end 420 can be sharp. For example, the distal end 420 can be used for piercing target tissue. In some instances, the sharp distal end can be opposingly oriented around a circumference of the cannula shaft 412 relative to the curved and/or rounded portion 430. The infusion cannula 400 can comprise a cannula hub having a hub lumen extending therethrough. The cannula shaft 412 extending distally from the cannula hub. The hub lumen can be in fluid communication with the infusion lumen 440 extending through the cannula shaft 412. In some instances, the infusion cannula 400 can be used with one or more cannula catheters described herein, including the cannula catheter 300 described with reference to FIGS. 1A, 1B, 2A, 2B, 3A, 3B and 3C. In some instances, a medical infusion system can comprise the infusion cannula 400. For example, the medical infusion system can comprise one or more other features of the medical infusion system 200 described with reference to FIGS. 1A, 1B, 2A, 2B, 3A, 3B and 3C.

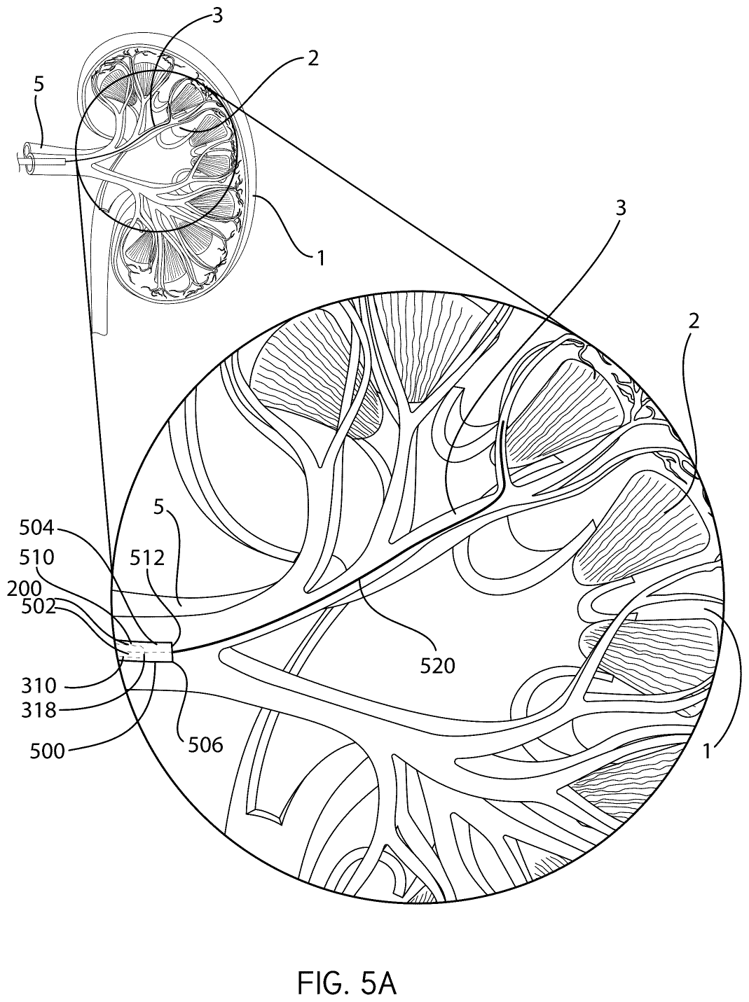

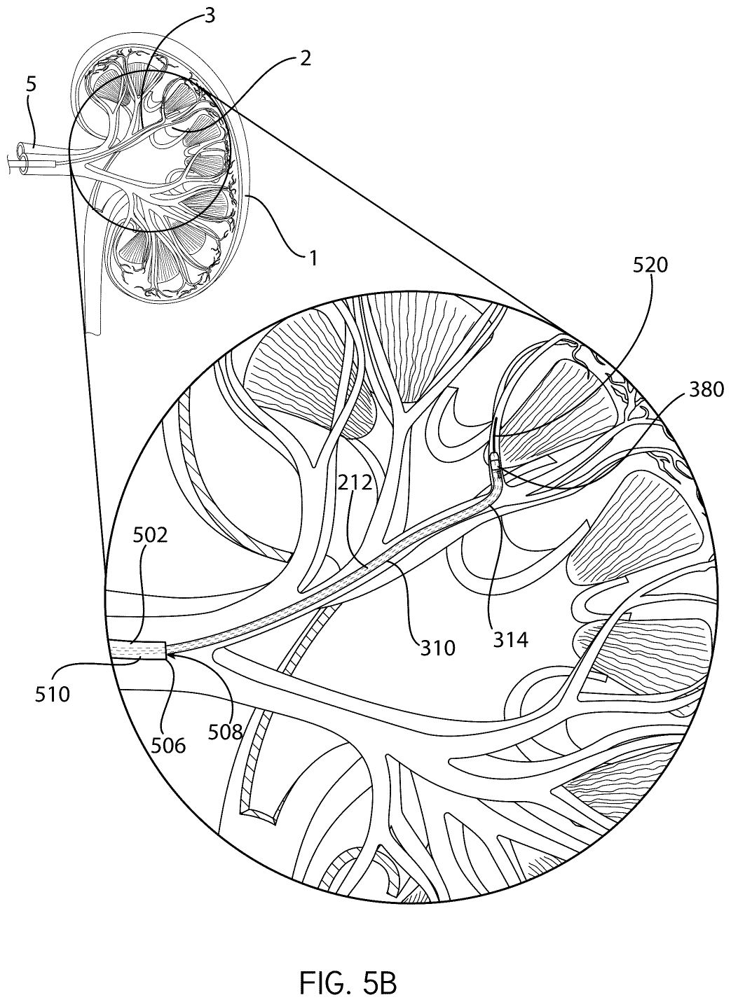

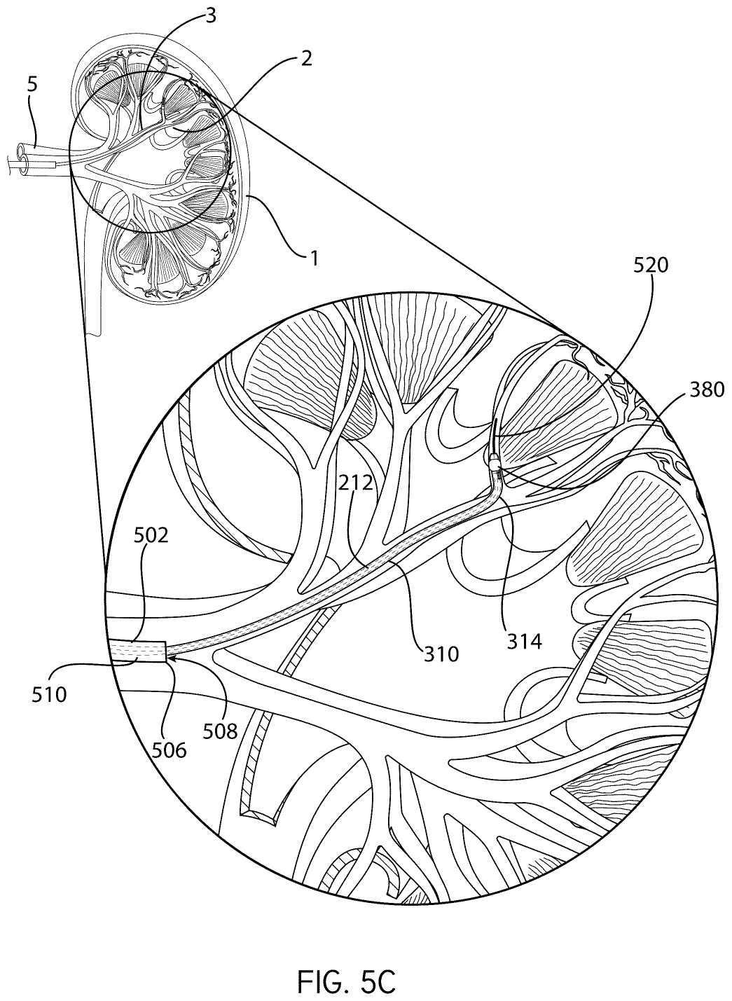

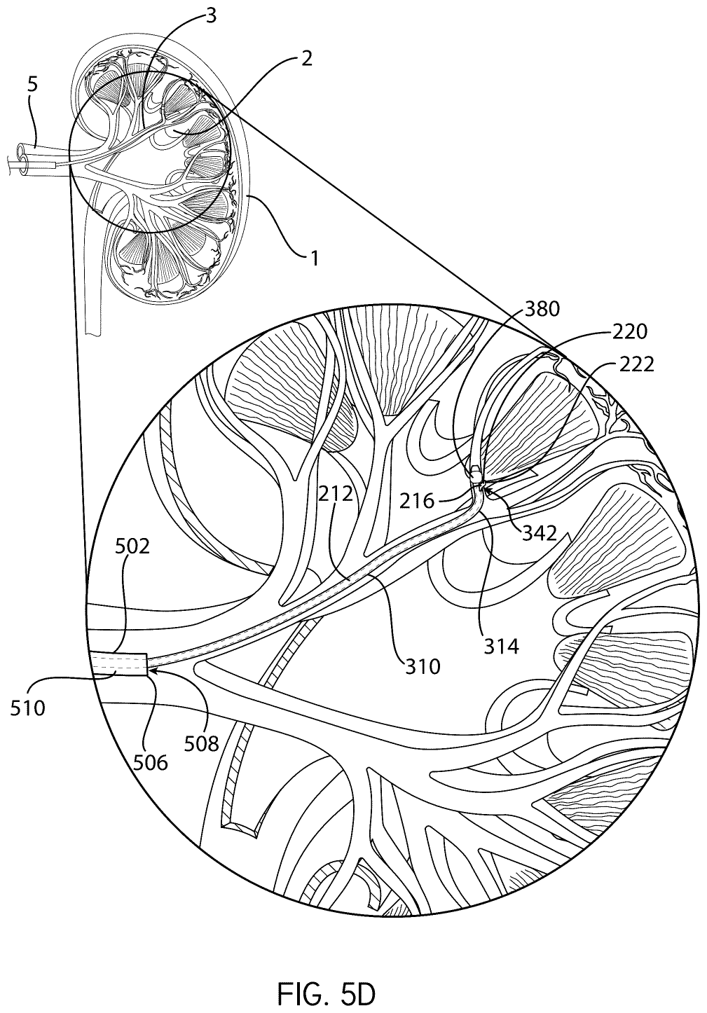

In some instances, a delivery catheter can be used to facilitate delivery of a medical infusion system to a target location. FIGS. 5A, 5B, 5C and 5D show various steps of an example of a process for providing an infusion to a target site within an inner renal medulla 2 of a kidney 1 using the medical infusion system 200 described with reference to FIGS. 1A, 1B, 2A, 2B, 3A, 3B and 3C, where a delivery catheter 500 is used to facilitate positioning of the medical infusion system 200. In some alternative instances, it will be understood that a medical infusion system comprising an infusion cannula 400 described with reference to FIG. 4 can be used for providing the infusion to the target site. Referring to FIG. 5A, the delivery catheter 500 carrying the medical infusion system 200 is shown as being positioned into the renal vein 5 along a guidewire 520. The delivery catheter 500 can comprise a delivery catheter shaft 502 comprising a delivery lumen 510 extending therethrough. The catheter shaft 310 having the cannula shaft 212 (not shown) preloaded therein can comprise at least a portion disposed within the delivery lumen 510. In some instances, the distal end 318 of the catheter shaft 310 can be within a distal portion 504 of the delivery catheter shaft 502. For example, the distal end 318 of the catheter shaft 310 can be disposed within a distal portion 512 of the delivery lumen 510. In some instances, the distal end 318 of the catheter shaft 310 can be at or proximate to the distal end 506 of the delivery catheter shaft 502.

The renal vein 5 can be accessed via a femoral vein. In some instances, a delivery path for positioning a portion of the catheter shaft 310 into the kidney 1 can comprise inserting the portion of the catheter shaft 310 through an opening formed on the femoral vein and advancing the portion of the catheter shaft 310 through the femoral vein. The portion of the catheter shaft 310 can be advanced into an inferior vena cava, and then into the renal vein 5. For example, the guidewire 520 can be positioned along the delivery path. The delivery catheter shaft 502 carrying the catheter shaft 310 and the cannula shaft 212 can be advanced along the guidewire 520 into the renal vein 5. For example, a portion of the delivery catheter shaft 502 carrying the catheter shaft 310 and the cannula shaft 212 can be inserted into an access opening formed on the femoral vein and advanced through the femoral vein. The portion of the delivery catheter shaft 502 can be advanced into the inferior vena cava, and then into the renal vein 5. The distal portion 504 of the delivery catheter shaft 502 can be positioned into the renal vein 5. For example, the distal end 506 of the delivery catheter shaft 502 can be positioned into and remain in the renal vein 5.

In FIG. 5B, a portion of the catheter shaft 310 is shown as being disposed distally of the distal end 506 of the delivery catheter shaft 502. For example, after the distal portion 504 of the delivery catheter shaft 502 is desirably positioned within the renal vein 5, a portion of the catheter shaft 310 can be advanced out of a distal opening 508 at the distal end 506 of the delivery catheter shaft 502. The catheter shaft 310 carrying the cannula shaft 212 can be advanced relative to the delivery catheter shaft 502 to deploy the catheter shaft 310. The inflatable balloon 380 disposed around the distal portion 314 of the catheter shaft 310 can be in a deflated state while the catheter shaft 310 is advanced to a target location. For example, FIG. 5B shows the inflatable balloon 380 in the deflated state. The catheter shaft 310 can be advanced until the side opening 342 of the catheter shaft 310 is disposed distally of the distal end 506 of the delivery catheter shaft 502. In some instances, the catheter shaft 310 can be advanced to position the side opening 342 at a target location in the inner medulla 2. In some instances, a portion of the catheter shaft 310 can be advanced into a venule vessel 3 within the inner medulla 2. For example, the distal portion 314 of the catheter shaft 310 can be positioned into the venule vessel 3 such that the side opening 342 is positioned at a target location in the venule vessel 3. As described herein, the target location within the venule vessel 3 can be at a location adjacent to interstitium tissue surrounding, adjacent and/or proximate to the loop of Henle. The catheter shaft 310 can be advanced while the inflatable balloon 380 is in the deflated state until the side opening 342 is at the target location in the venule vessel 3.

As shown in FIG. 5C, the inflatable balloon 380 disposed around the catheter shaft 310 can be inflated after the catheter shaft 310 is at the desired location within the venule vessel 3. The inflatable balloon 380 is shown in an inflated state in FIG. 5C. The inflatable balloon 380 can engage with respective portions of the venule vessel 3 to facilitate anchoring the distal portion 314 of the catheter shaft 310 at the target location. Engagement of the inflatable balloon 380 with the portions of the venule vessel 3 can fix or substantially fix the position of the distal portion 314 of the catheter shaft 310. In some instances, engagement of the inflatable balloon 380 with corresponding portions of the venule vessel wall 3 can prevent or reduce distal movement of the catheter shaft 310 during deployment of the cannula shaft 212 from the catheter shaft 310 and/or delivery of infusion fluid through the cannula shaft 212.

Referring to FIG. 5D, a portion of the cannula shaft 212 is shown as being disposed externally of the catheter shaft 310. As described herein, the cannula shaft 212 can be preloaded within the catheter shaft 310 while the catheter shaft 310 is advanced along the delivery path. For example, while the cannula shaft 212 is preloaded, the distal end 220 of the cannula shaft 212 can be adjacent to the side opening 342 of the catheter shaft 310. In some instances, after the catheter shaft 310 is desirably positioned, the cannula shaft 212 can be advanced distally relative to the catheter shaft 310 to deploy a portion of the cannula shaft 212 through the side opening 342. The distal portion 216 of the cannula shaft 212 can be advanced through the side opening 342 such that the distal end 220 of the cannula shaft 212 can puncture a portion of the wall of the venule vessel 3. The cannula shaft 212 can be advanced relative to the catheter shaft 310 until the distal opening 222 of the cannula shaft 212 is positioned through the wall of the venule vessel 3. The portion of the cannula shaft 212, including the distal opening 222, can be positioned into the interstitium tissue adjacent to the venule vessel 3. The infusion fluid can subsequently be provided through the cannula shaft 212 into the interstitium tissue after the cannula shaft 212 is desirably positioned.

The infusion fluid can be delivered to the target interstitium tissue in the inner renal medulla 2 to increase the osmotic pressure in the target interstitium tissue. The osmotic pressure can be increased to achieve desired reduction in water reabsorption. Increasing the osmotic pressure within the interstitium tissue in the inner renal medulla 2, including interstitium tissue in the inner renal medulla 2 adjacent and/or proximate to, and/or surrounding, the loop of Henle, can advantageously decrease water reabsorption from the loop of Henle, reducing water reabsorption within the kidney. In some instances, infusion fluid can be delivered to the target interstitium tissue in the inner renal medulla 2 can be performed until an osmotic pressure of greater than that in the loop of Henle. In some instances, the infusion fluid can be provided until the osmotic pressure within the target interstitium tissue is greater than about 1200 milliosmoles per liter (mOsm/L). In some instances, the desired osmotic pressure in the target interstitium tissue can be between about 1250 milliosmoles per liter (mOsm/L) and about 1450 milliosmoles per liter (mOsm/L). In some instances, the desired osmotic pressure in the target interstitium tissue can be greater than about 1450 milliosmoles per liter (mOsm/L). The rate of infusion can be selected to provide the desired change in osmotic pressure while reducing or avoiding undesired trauma to the patient. A variety of infusion rates may be applicable. In some instances, the infusion rate can be about 200 microliter per millimeter (u L/mm) of travel to about 300 microliter per millimeter (μL/mm) of travel. In some instances, the infusion rate can be about 250 microliter per millimeter (μL/mm) of travel. In some instances, the infusion rate can be less than about 200 microliter per millimeter (μL/mm) of travel. In some instances, the infusion rate can be greater than about 300 microliter per millimeter (μL/mm) of travel.

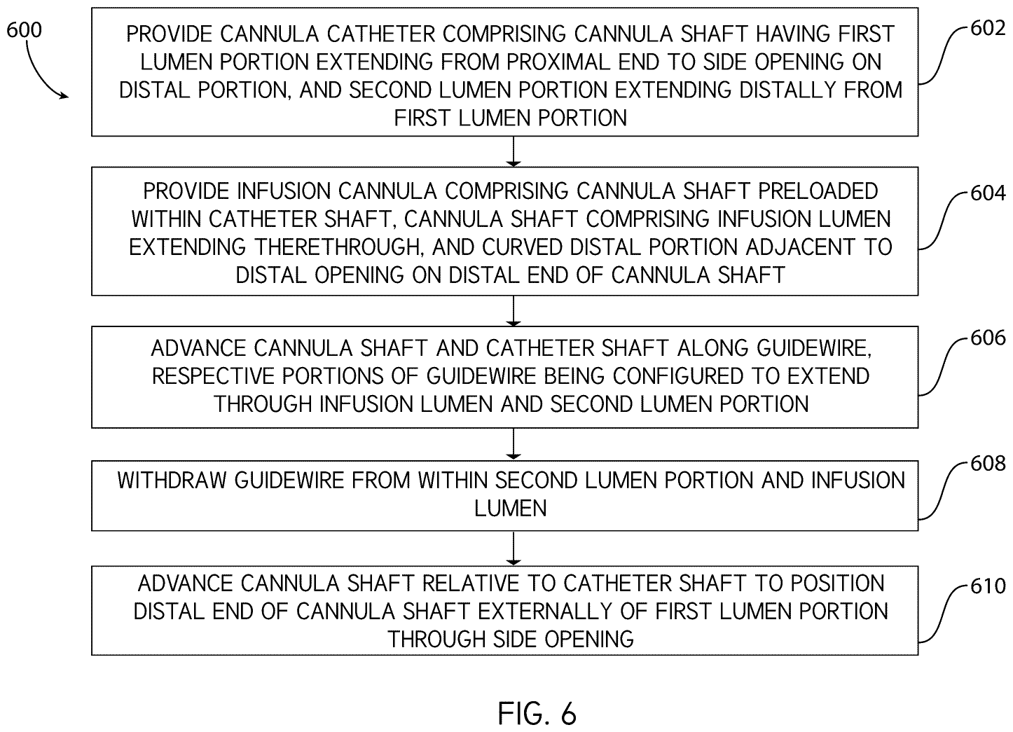

FIG. 6 is a process flow diagram of an example of a process 600 to provide an infusion to a target site using one or more medical infusion systems described herein, including for example, the medical infusion system 200 described with reference to FIGS. 1A, 1B, 2A, 2B, 3A, 3B and 3C. In some alternative instances, a medical infusion system comprising the infusion cannula 400 described with reference to FIG. 4 can be used. In block 602, the process can involve providing a cannula catheter comprising a cannula shaft. The cannula shaft can comprise a first lumen portion extending from a proximal end of the catheter shaft to a side opening on a distal portion of the catheter shaft. A second lumen portion can extend distally from the first lumen portion to a distal end of the catheter shaft. In block 604, the process can involve providing an infusion cannula that includes a cannula shaft. The cannula shaft can be preloaded within the catheter shaft. The cannula shaft can comprise an infusion lumen extending therethrough. The cannula shaft can comprise a curved distal portion adjacent to a distal opening on a distal end of the cannula shaft. The distal end of the cannula shaft can be disposed at or proximal to the side opening of the catheter shaft. In block 606, the process can involve advancing the cannula shaft and the catheter shaft along a guidewire, respective portions of the guidewire being configured to extend through the infusion lumen, the distal opening adjacent to the curved distal portion of the cannula shaft, the opening on the curved portion of the catheter shaft and the second lumen portion. In block 608, the process can involve withdrawing the guidewire from within the second lumen portion and the infusion lumen. In block 610, the process can involve advancing the cannula shaft relative to the catheter shaft to position the distal end of the cannula shaft externally of the first lumen portion through the side opening.

The cannula shaft and the catheter shaft can be advanced along a guidewire disposed along a transvascular delivery path to a target position within a blood vessel of the inner medulla. The blood vessel can be a direct or indirect branch of the renal vein. In some instances, the target location can be within a venule vessel of the inner renal medulla. The cannula shaft can be used to puncture a wall portion of the blood vessel such that a portion of the cannula shaft can be advanced into tissue adjacent to the blood vessel to provide the infusion. In some instances, advancing the cannula shaft can comprise puncturing the target tissue using the distal end of the cannula shaft and positioning the distal opening of the cannula shaft into the target tissue. In some instances, the process of providing an infusion can involve infusing a fluid into the target tissue through the infusion lumen. The fluid can be delivered to the target tissue through the distal opening.

In some instances, advancing the cannula shaft and the catheter shaft can comprise advancing a distal portion of the catheter shaft and the distal portion of the cannula shaft into a venule vessel of an inner renal medulla. After the distal portion of the catheter shaft and the distal portion of the cannula shaft are at a desired position within the venule vessel, the cannula shaft can be advanced relative to the catheter shaft to puncture a wall of the venule vessel. Advancing the cannula shaft can comprise puncturing the wall of the venule vessel and positioning the distal opening adjacent to the curved portion of the catheter shaft into interstitium tissue of the inner renal medulla.

As described herein, an inflatable balloon can be coupled to the distal portion of the catheter shaft. The inflatable balloon can be disposed around the distal portion of the catheter shaft. The inflatable balloon can be inflated to anchor the distal portion of the catheter shaft at the target location within the blood vessel. In some instances, after the distal portion of the catheter shaft is at the desired position within the venule vessel, the inflatable balloon can be inflated to anchor the distal portion of the catheter shaft within the venule vessel.

In some instances, the process can involve infusing the interstitium tissue adjacent to the blood vessel at the target location with an oncotically active solution. The oncotically active solution can comprise at least one of albumin, dextran, and mannitol. In some instances, infusing the interstitium tissue can comprise manipulating a dial syringe coupled to a proximal portion of the infusion cannula to control fluid flow through the infusion cannula. The dial syringe can be configured to facilitate desired control in flowrate of the fluid into the inner medulla to provide desired change in osmotic pressure of the interstitium tissue while reducing or preventing undesired trauma to the patient.

In some instances, a delivery catheter can be used to facilitate delivery of the cannula shaft and catheter shaft to the target site. The delivery catheter can comprise a delivery lumen extending therethrough. In some instances, providing the catheter shaft and the cannula shaft can comprise providing the catheter shaft and the cannula shaft preloaded within the delivery lumen of the delivery catheter. In some instances, advancing the catheter shaft and the cannula shaft along the guidewire can comprise advancing the delivery catheter carrying the catheter shaft and the cannula shaft along the guidewire.

The delivery catheter can be advanced to a first location along the transvascular delivery path to the target location in the inner renal medulla. In some instances, advancing the delivery catheter can comprise advancing the delivery catheter carrying the catheter shaft and the cannula shaft to a renal vein. The catheter shaft carrying the cannula shaft preloaded therein can then be deployed from the delivery catheter at the first location, for example at the location in the renal vein. The catheter shaft can be advanced relative to the delivery catheter to position at least a portion of the catheter shaft distally of a distal opening at a distal end of the delivery catheter and into the renal vein. The catheter shaft and cannula shaft can subsequently be advanced to the target location within a blood vessel in the inner medulla. In some instances, a portion of the catheter shaft can be advanced into a target location in a venule vessel. A portion of the cannula shaft can be advanced out of the side opening of the catheter shaft to puncture the venule vessel. The cannula shaft can be advanced relative to the catheter shaft to position the distal end and the distal opening of the cannula shaft at a desired location in the renal tissue.

In some instances, providing the cannula catheter can comprise providing a catheter hub comprising a cannula advancement assembly configured to engage with the cannula shaft to control translational movement of the cannula shaft. The catheter hub can comprise a first catheter hub portion and a second catheter hub portion. Advancing the cannula shaft relative to the catheter shaft can comprise rotating the first catheter hub portion around a longitudinal axis of the catheter hub relative to the second catheter hub portion of the catheter hub in a first direction to cause the cannula advancement assembly to advance the cannula shaft. In some instances, rotating the first catheter hub portion relative to the second catheter hub portion in a second opposing direction can be configured to cause the cannula advancement assembly to retract the cannula shaft. For example, the first catheter hub portion can be rotated around the longitudinal axis in the second opposing direction until the cannula shaft is retracted back into the catheter shaft after the infusion is completed and prior to withdrawing the catheter shaft and cannula shaft from the patient.

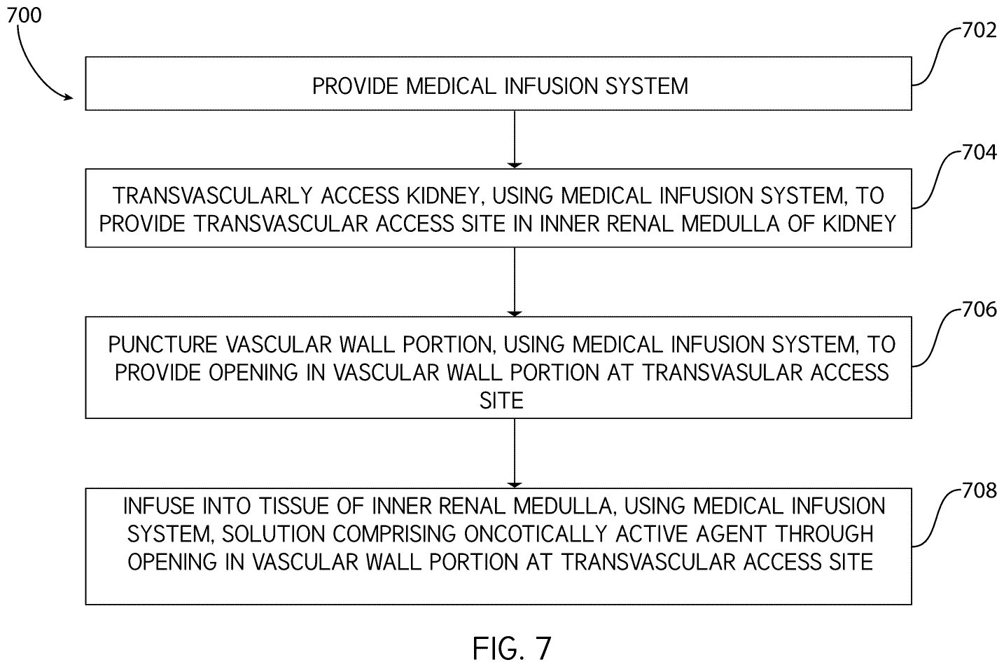

FIG. 7 is a process flow diagram of an example of process 700 for providing an infusion. In some instances, a medical infusion system comprising one or more features described herein can be used to provide the infusion, including for example, the medical infusion system 200 described with reference to FIGS. 1A, 1B, 2A, 2B, 3A, 3B and 3C. In some alternative instances, a medical infusion system comprising the infusion cannula 400 described with reference to FIG. 4 can be used. In block 702, the process can involve providing a medical infusion system. In block 704, the process can involve transvascularly accessing a kidney, using the medical infusion system, to provide a transvascular access site in an inner renal medulla of the kidney. In block 706, the process can involve puncturing a vascular wall portion, using the medical infusion system, to provide an opening in the vascular wall portion at the transvasular access site. In block 708, the process can involve infusing into tissue of the inner renal medulla, using the medical infusion system, a solution comprising an oncotically active agent through the opening in the vascular wall portion at the transvascular access site.

Infusing into tissue of the inner renal medulla can be configured to modify the osmotic pressure within the inner renal medulla of the kidney. Modifying the osmotic pressure within the inner medulla of the kidney can advantageously decrease water reabsorption in the kidney. A decrease in reabsorption of water can lead to an increase in urine output and a decrease in blood volume. Decreased blood volume can lead to reduced renal venous pressure to facilitate an increase in the renal pressure gradient. Increasing the renal pressure gradient can improve renal filtration function. Improved renal filtration function can facilitate improvement in acute decompensated heart failure (ADHF), such as in cardiorenal syndrome patients. In some instances, modifying the osmotic pressure of interstitium tissue surrounding, proximate and/or adjacent to one or more portions of the loop of Henle can advantageously effect the desired change in osmotic pressure within the inner medulla. In some instances, transvascularly accessing the kidney can comprise transvascularly accessing the kidney to provide a transvascular access to interstitium tissue in the inner renal medulla surrounding, proximate and/or adjacent to the loop of Henle.

In some instances, transvascularly accessing the inner medulla can comprise accessing the inner medulla using a renal vein. For example, a target location of interstitium tissue in the inner medulla can be accessed through the renal vein. In some instances, the renal vein can be accessed in a minimally invasive percutaneous transcatheter procedure through a femoral vein. One or more portions of the medical infusion system as described herein can be inserted into the femoral vein. The one or more portions of the medical infusion system can be advanced through the femoral vein and into an inferior vena cava. In some instances, the one or more portions of the medical infusion system can be advanced from the inferior vena cava into the renal vein. The one or more portions of the medical infusion system can be advanced to a target location within the inner renal medulla. A target location within the inner renal medulla can be a location in a blood vessel which is a direct or indirect branch of the renal vein. The target location can be in a portion of the blood vessel adjacent to the desired interstitium tissue in the inner medulla. In some instances, accessing the inner medulla can be through a venule vessel within the inner medulla. In some instances, transvascularly accessing the kidney can comprise transvascularly accessing the kidney to provide a transvascular access in a venule vessel of the kidney. The venule vessel in the renal medulla can be accessed through the renal vein. For example, the one or more portions of the medical infusion system can be advanced through the renal vein and subsequently into the venule vessel.

A wall portion of the blood vessel at the target location can be punctured to allow advancement therethrough of a portion of the medical infusion system such that the infusion fluid can be provided into the interstitium tissue adjacent to the blood vessel through the medical infusion system. In some instances, a wall portion of a blood vessel that is a direct or indirect branch of the renal vein can be punctured. In some instances, puncturing the vascular wall portion can comprise puncturing a wall portion of the venule vessel to provide an infusion site. In some instances, infusing into the tissue of the inner renal medulla can comprise infusing the solution into interstitium tissue of the inner renal medulla adjacent to the infusion site.

In some instances, the oncotically active agent can comprise a biocompatible, biologically inert and/or non-resorbable agent. For example, the oncotically active agent can be a biocompatible, biologically inert and non-resorbable agent. In some instances, the oncotically active agent can comprise at least one of albumin, dextran, and mannitol. Infusing into tissue of the inner renal medulla the solution comprising the oncotically active agent can comprise infusing a solution comprising one or more of albumin, dextran, and mannitol. The infusion can be provided until a desired osmotic pressure is achieved in the target interstitium tissue of the inner medulla. As described herein, the target interstitium tissue can be interstitium tissue surrounding, proximate and/or adjacent to the loop of Henle. The desired osmotic pressure can be an osmotic pressure higher than that in the loop of Henle. For example, the osmotic pressure in the loop of Henle can be about 1200 milliosmoles per liter (mOsm/L). In some instances, infusing into the tissue of the renal medulla can comprise providing an osmotic pressure of greater than about 1200 milliosmoles per liter (mOsm/L) in the target interstitium tissue. In some instances, the desired osmotic pressure in the target interstitium tissue can be between about 1250 milliosmoles per liter (mOsm/L) and about 1450 milliosmoles per liter (mOsm/L). In some instances, the desired osmotic pressure in the target interstitium tissue can be greater than about 1450 milliosmoles per liter (mOsm/L). The rate of infusion can be selected to provide the desired change in osmotic pressure while reducing or avoiding undesired trauma to the patient. In some instances, infusing into the tissue can comprise infusing into the tissue with the solution at a rate of about 200 microliter per millimeter (μL/mm) of travel to about 300 microliter per millimeter (μL/mm) of travel. In some instances, infusing into the tissue can comprise infusing into the tissue with the solution at a rate of about 250 microliter per millimeter (μL/mm) of travel.

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below.

Example 1: A medical infusion system comprising an infusion cannula comprising a cannula shaft having an infusion lumen extending therethrough that is configured to slidably receive a guidewire, and a cannula catheter comprising a catheter shaft having a catheter lumen extending therethrough. The catheter lumen can comprise a first lumen portion extending from a proximal end of the catheter shaft to a side opening on a distal portion of the catheter shaft, at least a portion of the cannula shaft being configured to be slidably disposed in the first lumen portion and a distal portion of the cannula shaft being configured to be advanced out of the first lumen portion through the side opening. The catheter lumen can comprise a second lumen portion extending distally from the first lumen portion to a distal end of the catheter shaft, the second lumen portion being configured to slidably receive the guidewire.

Example 2: The system of any example herein, in particular example 1, wherein the distal portion of the cannula shaft comprises a curved portion.

Example 3: The system of any example herein, in particular example 2, wherein the cannula shaft comprises a distal opening adjacent to the curved portion of the cannula shaft, and wherein the guidewire is configured to extend through the distal opening from the infusion lumen into the second lumen portion.

Example 4: The system of any example herein, in particular examples 1 to 3, wherein at least a portion of the infusion cannula is configured to be preloaded within the first lumen portion, and the distal portion of the cannula shaft comprises a curved portion and a distal opening adjacent to the curved portion of the cannula shaft. While the infusion cannula is preloaded within the first lumen portion, the corresponding portion of the guidewire can be configured to be slidably disposed within the infusion lumen from a proximal end of the infusion lumen to the distal opening adjacent to the curved portion, and the guidewire can be configured to extend out of the distal opening adjacent to the curved portion and into the second lumen portion.

Example 5: The system of any example herein, in particular example 4, wherein the guidewire is configured to be withdrawn from the infusion lumen and the second lumen portion to allow advancement of a portion of the cannula shaft out of the catheter shaft through the side opening.

Example 6: The system of any example herein, in particular example 4 or 5, wherein, while the infusion cannula is preloaded within the first lumen portion, the curved portion of the cannula shaft is configured to be positioned against a corresponding curved portion of an inner lumen wall adjacent to the side opening defining the first lumen portion.

Example 7: The system of any example herein, in particular examples 4 to 6, wherein, while the cannula shaft is preloaded within the first lumen portion, the distal opening adjacent to the curved portion of the cannula shaft is on a longitudinal axis of the cannula catheter.

Example 8: The system of any example herein, in particular examples 4 to 7, wherein the catheter shaft comprises a distal opening at the distal end of the catheter shaft and in fluid communication with the second lumen portion, and the guidewire is configured to extend through the distal opening and out of the second lumen portion.

Example 9: The system of any example herein, in particular examples 4 to 8, wherein the cannula shaft comprises a sharp distal end configured to pierce tissue, and the sharp distal end is configured to be adjacent to the side opening of the catheter shaft.

Example 10: The system of any example herein, in particular examples 4 to 9, wherein the infusion lumen is configured to be coaxial with a proximal portion of the first lumen portion.

Example 11: The system of any example herein, in particular examples 4 to 10, wherein longitudinal axes of the infusion lumen and the proximal portion of the first lumen portion are coaxial with a longitudinal axis of the cannula catheter.

Example 12: The system of any example herein, in particular examples 1 to 11, wherein at least a portion of an externally oriented surface of the cannula shaft is configured to be in contact with a corresponding portion of an inner lumen wall defining the first lumen portion.

Example 13: The system of any example herein, in particular example 12, wherein externally oriented surface portions of the cannula shaft received by the first lumen portion are configured to be in contact with corresponding portions of the inner lumen wall of the first lumen portion.

Example 14: The system of any example herein, in particular example 12 or 13, wherein the at least a portion of the externally oriented surface of the cannula shaft comprises a plurality of patterned indentations.

Example 15: The system of any example herein, in particular examples 1 to 14, wherein the second lumen portion comprises a diameter smaller than that of the first lumen portion.

Example 16: The system of any example herein, in particular examples 1 to 15, wherein a wall portion of the catheter shaft defining the second lumen portion is thicker than a wall portion of the catheter shaft defining the first lumen portion.

Example 17: The system of any example herein, in particular examples 1 to 16 further comprising an inflatable balloon coupled to an externally oriented surface portion on a distal portion of the catheter shaft, and an inflation lumen extending along a length of the catheter shaft and in fluid communication with the inflation balloon.

Example 18: The system of any example herein, in particular example 17, wherein the inflatable balloon is distal of the side opening.

Example 19: The system of any example herein, in particular example 17, wherein the inflatable balloon is proximal of the side opening.

Example 20: The system of any example herein, in particular examples 17 to 19, wherein the inflation lumen extends along a wall portion of the catheter shaft.

Example 21: The system of any example herein, in particular examples 17 to 20, wherein the inflatable balloon is around a circumference of the distal portion of the catheter shaft.

Example 22: The system of any example herein, in particular examples 1 to 21, wherein an outer diameter of the catheter shaft is less than about 2 French (Fr).

Example 23: The system of any example herein, in particular examples 1 to 22, wherein the infusion system is configured to be transvascularly advanced into an inner renal medulla to deliver a solution comprising an oncotically active agent.

Example 24: The system of any example herein, in particular examples 1 to 23, wherein the cannula catheter comprises a catheter hub comprising a cannula advancement assembly configured to engage with the infusion cannula to control translational movement of the infusion cannula. The system can include a first catheter hub portion rotatable around a longitudinal axis of the catheter hub relative to a second catheter hub portion of the catheter hub, wherein rotation of the first catheter hub portion relative to the second catheter hub portion in a first direction is configured to cause the cannula advancement assembly to translate the cannula shaft distally relative to the catheter shaft and rotation in a second opposing direction is configured to cause the cannula advancement assembly to translate the cannula shaft proximally relative to the catheter shaft.

Example 25: The system of any example herein, in particular examples 1 to 24 further comprising a dial syringe coupled to a cannula hub of the infusion cannula, the dial syringe being configured to control fluid flow through the infusion cannula.

Example 26: The system of any example herein, in particular examples 1 to 25, wherein the cannula shaft is a stainless-steel infusion cannula.

Example 27: The system of any example herein, in particular examples 1 to 26, wherein the catheter shaft is a polymeric catheter.

Example 28: The system of any example herein, in particular examples 1 to 27, wherein the infusion cannula and the cannula catheter are sterilized.

Example 29: A method of providing an infusion, the method comprising providing a cannula catheter comprising a cannula shaft, the cannula shaft comprising a first lumen portion extending from a proximal end of the catheter shaft to a side opening on a distal portion of the catheter shaft, and a second lumen portion extending distally from the first lumen portion to a distal end of the catheter shaft. The method can include providing an infusion cannula comprising a cannula shaft preloaded within the catheter shaft, the cannula shaft comprising an infusion lumen extending therethrough, and a curved distal portion adjacent to a distal opening on a distal end of the cannula shaft, and advancing the cannula shaft and the catheter shaft along a guidewire, respective portions of the guidewire being configured to extend through the infusion lumen, the opening on the curved distal portion of the cannula shaft, and the second lumen portion. The method can include withdrawing the guidewire from within the second lumen portion and the infusion lumen, and advancing the cannula shaft relative to the catheter shaft to position a distal end of the cannula shaft externally of the first lumen portion through the side opening.

Example 30: The method of any example herein, in particular example 29, wherein advancing the cannula shaft comprises puncturing target tissue using the distal end of the cannula shaft and positioning the distal opening adjacent to the curved distal portion into the target tissue.

Example 31: The method of any example herein, in particular example 30, further comprising infusing a fluid into the target tissue through the infusion lumen.

Example 32: The method of any example herein, in particular examples 29 to 31, wherein advancing the cannula shaft and the catheter shaft comprises advancing a distal portion of the cannula shaft and the distal portion of the cannula shaft into a venule vessel of an inner renal medulla.

Example 33: The method of any example herein, in particular example 32, wherein advancing the cannula shaft relative to the catheter shaft comprises puncturing a wall of the venule vessel and positioning the distal opening adjacent to the curved portion of the catheter shaft into interstitium tissue of the inner renal medulla.

Example 34: The method of any example herein, in particular example 33, further comprising infusing the interstitium tissue with an oncotically active solution.

Example 35: The method of any example herein, in particular example 34, wherein infusing the interstitium tissue comprises infusing the interstitium tissue with an oncotically active solution comprising at least one of albumin, dextran, and mannitol.

Example 36: The method of any example herein, in particular example 34 or 35, wherein infusing the interstitium tissue comprises manipulating a dial syringe coupled to a proximal portion of the infusion cannula to control fluid flow through the infusion cannula.

Example 37: The method of any example herein, in particular examples 32 to 36, further comprising inflating an inflatable balloon disposed around the distal portion of the catheter shaft to anchor the distal portion of the catheter shaft within the venule vessel.

Example 38: The method of any example herein, in particular examples 29 to 37, further comprising providing a delivery catheter comprising a delivery lumen extending therethrough, and wherein providing the catheter shaft and the cannula shaft comprises providing the catheter shaft and the cannula shaft preloaded within the delivery lumen, and advancing the catheter shaft and the cannula shaft along the guidewire comprises advancing the delivery catheter carrying the catheter shaft and cannula shaft along the guidewire.

Example 39: The method of any example herein, in particular example 38, wherein advancing the delivery catheter comprises advancing the delivery catheter carrying the catheter shaft and the cannula shaft to a renal vein, and advancing the catheter shaft relative to the delivery catheter to position at least a portion of the catheter shaft distally of a distal opening at a distal end of the delivery catheter and into the renal vein.

Example 40: The method of any example herein, in particular example 39, further comprising advancing a portion of the catheter shaft into a target location in a venule vessel and advancing a portion of the cannula shaft out of the side opening of the catheter shaft to puncture the venule vessel.

Example 41: The method of any example herein, in particular examples 29 to 40, wherein providing the cannula catheter comprises providing a catheter hub comprising a cannula advancement assembly configured to engage with the cannula shaft to control translational movement of the cannula shaft, and wherein advancing the cannula shaft comprises rotating a first catheter hub portion around a longitudinal axis of the catheter hub relative to a second catheter hub portion of the catheter hub in a first direction to cause the cannula advancement assembly to advance the cannula shaft.

Example 42: The method of any example herein, in particular example 41, further comprising rotating the first catheter hub portion relative to the second catheter hub portion in a second opposing direction to cause the cannula advancement assembly to retract the cannula shaft back into the catheter shaft.

The above method(s) can be performed on a living animal or on a simulation, such as on a cadaver, cadaver heart, anthropomorphic ghost, simulator (e.g., with body parts, heart, tissue, etc. being simulated).

Example 43: A method of providing an infusion, the method comprising providing a medical infusion system, transvascularly accessing a kidney, using the medical infusion system, to provide a transvascular access site in an inner renal medulla of the kidney, puncturing a vascular wall portion, using the medical infusion system, to provide an opening in the vascular wall portion at the transvascular access site, and infusing into tissue of the inner renal medulla, using the medical infusion system, a solution comprising an oncotically active agent through the opening in the vascular wall portion at the transvascular access site.

Example 44: The method of any example herein, in particular example 43, wherein transvascularly accessing the kidney comprises transvascularly accessing the kidney to provide a transvascular access to interstitium tissue in the inner renal medulla adjacent to a loop of Henle.

Example 45: The method of any example herein, in particular example 43 or 44, wherein transvascularly accessing the kidney comprises transvascularly accessing the kidney to provide a transvascular access in a venule vessel of the kidney.

Example 46: The method of any example herein, in particular example 45, wherein puncturing the vascular wall portion comprises puncturing a wall portion of the venule vessel to provide an infusion site.

Example 47: The method of any example herein, in particular examples 43 to 46, wherein infusing into the tissue of the inner renal medulla comprises infusing the solution into interstitium tissue of the inner renal medulla adjacent to the infusion site.

Example 48: The method of any example herein, in particular examples 43 to 47, wherein the oncotically active agent comprises a biocompatible, biologically inert and non-resorbable.

Example 49: The method of any example herein, in particular example 48, wherein the oncotically active agent comprises at least one of albumin, dextran, and mannitol.

Example 50: The method of any example herein, in particular examples 43 to 49, wherein infusing into the tissue of the renal medulla comprises providing an osmotic pressure of greater than about 1200 mOsm/L in the tissue.

Example 51: The method of any example herein, in particular example 50, wherein the osmotic pressure in the tissue is between about 1250 mOsm/L and about 1450 mOsm/L.

Example 52: The method of any example herein, in particular examples 43 to 51, wherein infusing into the tissue comprises infusing into the tissue with the solution at a rate of about 200 μL/mm of travel to about 300 μL/mm of travel.

Example 53: The method of any example herein, in particular example 52, wherein infusing into the tissue comprises infusing into the tissue with the solution at a rate of about 250 μL/mm of travel.

The above method(s) can be performed on a living animal or on a simulation, such as on a cadaver, cadaver heart, anthropomorphic ghost, simulator (e.g., with body parts, heart, tissue, etc. being simulated).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for case of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of providing an infusion, the method comprising:
providing a cannula catheter comprising a catheter shaft, the catheter shaft comprising:
a first lumen portion extending from a proximal end of the catheter shaft to a side opening on a distal portion of the catheter shaft, and
a second lumen portion extending distally from the first lumen portion to a distal end of the catheter shaft;
providing an infusion cannula comprising a cannula shaft preloaded within the catheter shaft, the cannula shaft comprising:
an infusion lumen extending therethrough, and
a rounded distal portion adjacent to a distal opening on a distal end of the cannula shaft;
advancing the cannula shaft and the catheter shaft along a guidewire, respective portions of the guidewire being configured to extend through the infusion lumen, the distal opening adjacent to the rounded distal portion of the cannula shaft, and the second lumen portion;
withdrawing the guidewire from within the second lumen portion and the infusion lumen; and
advancing the cannula shaft relative to the catheter shaft to position a distal end of the cannula shaft externally of the first lumen portion through the side opening.

2. The method of claim 1, wherein advancing the cannula shaft comprises puncturing target tissue using the distal end of the cannula shaft and positioning the distal opening adjacent to the rounded distal portion into the target tissue.

3. The method of claim 1, wherein advancing the cannula shaft and the catheter shaft comprises advancing a distal portion of the cannula shaft and the distal portion of the cannula shaft into a venule vessel of an inner renal medulla.

4. The method of claim 3, wherein advancing the cannula shaft relative to the catheter shaft comprises puncturing a wall of the venule vessel and positioning the distal opening adjacent to the rounded portion of the catheter shaft into interstitium tissue of the inner renal medulla.

5. The method of claim 4, further comprising infusing the interstitium tissue with an oncotically active solution comprising at least one of albumin, dextran, and mannitol.

6. The method of claim 3, further comprising inflating an inflatable balloon disposed around the distal portion of the catheter shaft to anchor the distal portion of the catheter shaft within the venule vessel.

7. The method of claim 1, further comprising providing a delivery catheter comprising a delivery lumen extending therethrough, and wherein:
providing the catheter shaft and the cannula shaft comprises providing the catheter shaft and the cannula shaft preloaded within the delivery lumen; and
advancing the catheter shaft and the cannula shaft along the guidewire comprises advancing the delivery catheter carrying the catheter shaft and cannula shaft along the guidewire.

8. The method of claim 7, wherein:
advancing the delivery catheter comprises advancing the delivery catheter carrying the catheter shaft and the cannula shaft to a renal vein; and
advancing the catheter shaft relative to the delivery catheter to position at least a portion of the catheter shaft distally of a distal opening at a distal end of the delivery catheter and into the renal vein.

9. The method of claim 8, further comprising advancing a portion of the catheter shaft into a target location in a venule vessel and advancing a portion of the cannula shaft out of the side opening of the catheter shaft to puncture the venule vessel.

10. The method claim 1, wherein providing the cannula catheter comprises providing a catheter hub comprising a cannula advancement assembly configured to engage with the cannula shaft to control translational movement of the cannula shaft, and wherein advancing the cannula shaft comprises rotating a first catheter hub portion around a longitudinal axis of the catheter hub relative to a second catheter hub portion of the catheter hub in a first direction to cause the cannula advancement assembly to advance the cannula shaft.

11. The method of claim 10, further comprising rotating the first catheter hub portion relative to the second catheter hub portion in a second opposing direction to cause the cannula advancement assembly to retract the cannula shaft back into the catheter shaft.

12. A method of providing an infusion, the method comprising:
providing a cannula catheter comprising a catheter shaft;
providing an infusion cannula comprising a cannula shaft, the cannula shaft being at least partially slidably disposed within a first lumen portion of the catheter shaft;
transvascularly advancing the catheter shaft carrying the cannula shaft to a renal vein; and
advancing the cannula shaft relative to the catheter shaft to position a distal end of the cannula shaft externally of the first lumen portion through a side opening of the catheter shaft to puncture a renal vein wall portion and access renal tissue.

13. The method of claim 12, wherein:
providing the cannula shaft comprises providing a cannula shaft comprising a rounded portion adjacent to a distal opening on a distal end of the cannula shaft; and
advancing the cannula shaft relative to the catheter shaft comprises gliding the rounded portion along a curved portion of an inner lumen wall defining the first lumen portion of the catheter shaft to guide an exit trajectory of the cannula shaft through the side opening.

14. The method of claim 12, further comprising infusing into the renal tissue a solution comprising an oncotically active agent.

15. A method of providing an infusion, the method comprising:
providing a medical infusion system;
transvascularly accessing a kidney, using the medical infusion system, to provide a transvascular access site in an inner renal medulla of the kidney;
puncturing a vascular wall portion, using the medical infusion system, to provide an opening in the vascular wall portion at the transvascular access site; and
infusing into tissue of the inner renal medulla, using the medical infusion system, a solution comprising an oncotically active agent through the opening in the vascular wall portion at the transvascular access site.

16. The method of claim 15, wherein transvascularly accessing the kidney comprises transvascularly accessing the kidney to provide a transvascular access to interstitium tissue in the inner renal medulla adjacent to a loop of Henle.

17. The method of claim 15, wherein transvascularly accessing the kidney comprises transvascularly accessing the kidney to provide a transvascular access in a venule vessel of the kidney.

18. The method of claim 17, wherein puncturing the vascular wall portion comprises puncturing a wall portion of the venule vessel to provide an infusion site.

19. The method of claim 15, wherein infusing into the tissue of the inner renal medulla comprises infusing the solution into interstitium tissue of the inner renal medulla adjacent to the infusion site.

20. The method of claim 15, wherein infusing into the tissue of the renal medulla comprises providing an osmotic pressure of about 1250 mOsm/L to about 1450 mOsm/L in the tissue.
